(19) United States
(12) Patent Application Publication
(10) Pub. No.: US 2016/0132122 A1
(43) Pub. Date: May 12, 2016

(54) Gesture Feedback for Non-Sterile Medical Displays

(71) Applicant: BRAINLAB AG, Feldkirchen (DE)

(72) Inventors: Wolfgang Steinle, Munich (DE); Nils Frielinghaus, Heimstetten (DE); Christoffer Hamilton, Aschheim (DE)

(73) Assignee: Brainlab AG, Feldkirchen (DE)

(21) Appl. No.: 14/894,313

(22) PCT Filed: May 29, 2013
(86) PCT No.: PCT/EP2013/061117

§ 371 Date: Nov. 25, 2015

(51) Int. Cl. G06F 3/01; A61B 5/00; G06F 3/0481; G06F 3/041; G06F 3/16

(52) U.S. Cl. CPC G06F 3/017; G06F 3/041; G06F 3/167; G06F 3/0481; A61B 5/7435; G06F 2203/04108; A61B 2017/00207

(57) ABSTRACT

The present disclosure relates to a feedback system for control or input gestures in a medical environment, which includes a presence detector which generates a threshold and acquires detection data which describe whether a medical input gesture reaches and/or crosses the threshold; and the extent to which the gesture has passed the threshold; and a computer which is connected to the presence detector and triggers a sterility-preserving response to a positive detection by the presence detector including information how dangerous the situation is becoming with regard to non-sterility. The invention also relates to a medical image display system comprising such a feedback system and to a method of providing sterility-preserving feedback with respect to control or input gestures in a medical system.

The invention relates to the general technical field of providing gesture feedback for control or input gestures in a medical environment.

In medical environments, in particular in operating theatres, it is vitally important to preserve sterility, i.e. to keep each and every person coming into contact with the patient sterile. On the other hand, personnel in operating theatres benefit to an ever increasing degree from technological medical equipment, and such medical equipment must of course be operated by said personnel. However, many of these devices cannot be sterilised or kept sterile and must be treated as non-sterile in general, such that any direct contact with such a device renders the respective person non-sterile.

One such device is for example a medical display such as a screen or monitor or a (digital) light box, used to display treatment assistance such as patient information or treatment guidance for a surgeon. Because it is not possible for these displays to be kept sterile, they are usually draped, but this reduces image quality. In the case of touch screens, the use of (switching) touch pens or gloves, foot switches and voice recognition has been proposed, but these solutions are often cumbersome or do not actually preserve sterility or are not suitable for controlling complex software.

Gesture recognition for providing screen-based or display-based inputs is known and described for example in EP 2 315 103 A2, U.S. Pat. No. 7,834,850 B2 or US 2007/0211031 A1. These methods of gesture recognition and “non-contact input” appear to solve the problem of medical devices which personnel cannot touch but must still control in their immediate vicinity. However, they do not actually provide a complete solution because they mostly require the user to make gestures in the immediate vicinity of the device, such as for example a computer monitor, which makes accidental contact and activation very likely to occur.

It is the general object of the present invention to preserve sterility in a medical environment in which control or input gestures have to be made in order to operate medical devices.

This object is achieved by a feedback system in accordance with claim 1, a medical image display system in accordance with claim 5 and a method of providing sterility-preserving feedback with respect to control or input gestures in a medical system in accordance with claim 9. The sub-claims define advantageous embodiments of the invention.

The feedback system in accordance with the present invention comprises:
a presence detector which generates a threshold and acquires detection data which describe whether a medical input gesture reaches and/or crosses the threshold; and
a computer which is connected (via a wire connection or wirelessly) to the presence detector and triggers a sterility-preserving response to a positive detection by the presence detector.

Sterility can advantageously be preserved in a medical environment, such as an operating theatre, by means of a feedback system according to the present invention. The invention utilises the realisation that providing non-contact operability for devices in a medical environment does not guarantee that non-sterile devices will not be touched by accident, thus rendering the touching person or object non-sterile. A presence detector which generates a threshold and a computer which triggers a sterility-preserving response in accordance with the invention make it possible to detect whether a non-sterile incidence is about to occur or has occurred, and the response generated opens up a range of possibilities for starting or triggering counter measures.

In most cases, the threshold will not be a physical threshold but is rather projected into the portion of space which is of interest or is based on monitoring the portion of space which is of interest using sensors. In such cases, the threshold could be described as a “virtual threshold” which has a function and an extension within the portion of space which is of interest but no actual bodily or physical presence.

In one embodiment, the presence detector comprises a sensor unit which monitors the presence of an object in a predetermined portion of space which includes or forms the threshold. It is conceivable for light sensor arrays or individual sensors in a form adapted to the respective application to be used as the presence detector. Combinations of active light-emitting units and sensor units can be used, i.e. either “active” or “passive” presence detectors may be suitable for applications involving the present invention.

Two or more image sensors may be used which consist of rows of light-sensing elements. Sensors of this type are known from photocopiers and/or scanners. These sensors would monitor the region of the threshold, typically at different angles and/or using different lenses, or a digital camera system could be used to focus on the position at which the object reaches or crosses the threshold. In embodiments of an active presence detector, light could be projected into the threshold area, for example in simple embodiments as a light barrier, photoelectric barrier or sensor barrier. Infrared light can be used for projecting and/or monitoring the threshold in order to avoid interference from light sources in the vicinity.

In one embodiment, the presence detector monitors a substantially planar sensing area which includes or forms a threshold plane. In this case, and where the application necessitates that unintentional contact with a substantially planar face is avoided, the sensors, sensor arrays or light projection devices are preferably arranged in a linear fashion or are linear sensor-projectors. In any event, LEDs could be used as the light emitters.

In another embodiment of the present invention, the computer is connected to a response indicator which issues a “sterility-preserving signal”, which is a very general way of describing how the knowledge of an imminent incidence of non-sterile contact would be used to avoid precisely such an incidence.

Another aspect of the present invention relates to a medical image display system comprising a feedback system such as has been described above in various possible embodiments. The medical image display system also comprises a medical display in front of which the threshold is generated. This aspect represents a very advantageous application of the present invention, since on the one hand, it meets the requirement of preserving sterility and on the other hand, it utilises the display as a control or input instrument. In other words, the present invention may be said to solve the conflict between the desire to use familiar and tried display controls, which tend to involve making gestures and/or moving gesturing objects directly in front of and near to the surface of a display, and the need to avoid the danger of non-sterility inherent in this close approach to the surface of the display. Providing the threshold and triggering a sterility-preserving response if the threshold in front of the display is reached or crossed enables such displays to be used with much greater confidence with regard to the issue of sterility.

In one embodiment, the display system of the present invention can use a threshold which is located at a predetermined or selectable distance from the display which represents a minimum distance to be observed when a gesture or gesturing object approaches the display. This ensures that the system knows when a certain minimum distance has been reached and there is a danger of non-sterility. Continuous or incremental distance measurements are of course also conceivable in other embodiments and may give more detailed information about how far the object still is from the display or how dangerous the situation is becoming with regard to non-sterility. The threshold need not be a strictly two-dimensional object extending in a flat plane but rather can also extend in a curved plane and/or can exhibit a certain thickness, such that it is possible to determine the extent to which a gesture or gesturing object has entered the threshold. The same can also be achieved by thresholds consisting of more than one flat (or curved) planes arranged consecutively in a direction perpendicular to their main direction of extension, in particular their plane of extension. The response triggered by the computer can of course be adapted to these respective continuous or incremental approaches to the threshold in front of the display.

One embodiment of the display system uses a positioning aid which assists in establishing and/or maintaining a predetermined or selectable distance between the medical display and the threshold and/or between the medical display and the presence detector. Such a positioning aid can simply be a bar which is or can be attached to a portion of the display and which extends in the direction in which the display is supposed to be arranged. The bar can have a predetermined length and/or can feature line markings in the manner of a ruler, such that a particular distance between the presence detector and the display can easily be predetermined when setting it up. In other possible embodiments, the positioning aid could comprise guides or guiding edges arranged at a predetermined distance from the display or could even comprise more sophisticated devices such as electro-optical length measurement devices, for example laser devices.

In another embodiment, the display system comprises a display moving unit for moving the display away from a gesture or gesturing object which has reached or crossed the threshold or breached a predetermined or selected distance from the display. While such systems would require motorised guide frames or rails or other moving systems to be installed, they would almost certainly prevent any unintentional contact with the display. It would in fact be necessary to deliberately follow the moving display in order to make “intentional” desterilising contact.

The method of the present invention provides a sterility-preserving feedback with respect to control or input gestures in a medical environment and comprises the steps of:
generating a threshold by means of a presence detector and acquiring detection data which describe whether a medical input gesture reaches and/or crosses the threshold; and
triggering a sterility-preserving response to a positive detection by the presence detector by means of a computer which is connected to the presence detector.

Some embodiments of the method of the invention use a feedback system in accordance with the embodiments described above and/or a display system in accordance with one or more of the embodiments described above.

It will be clear that the described advantages of the systems according to the present invention apply correspondingly to the method of the present invention in its various embodiments.

In one embodiment of the method according to the invention, the presence detector acquires data which describe the extent to which the gesture or gesturing object has crossed the threshold, in a continuous or non-continuous, in particular incremental, measurement. As already inferred above, such a feature could be used to establish a number of “danger levels” which reflect the increasing danger of non-sterility presented by ever closer approaches to the display. In accordance with another embodiment of the method according to the invention, the computer's response involves providing user guidance, in particular an audible or visible indication, signal or alarm, which in particular comprises one or more of the following:
a warning message, in particular if actual contact with the display has been detected by the presence detector or a touch screen display or a touch detector on the display;
a modification to the displayed image;
a view of the threshold area or a combined view of the threshold and display area which illustrates the extent to which the threshold or display has been approached.

Various forms of user guidance are conceivable in connection with this, and a wide range of possible warning messages, for example in different sizes and/or colours, are also conceivable.

The modification to the displayed image can take the form of one or more of the following changes to the display or the user interface shown on the display, which are made when the gesture or gesturing object touches or crosses the threshold and thus comes too close to the display.

One way of modifying the displayed image is to display a dangerous-looking object which the user would rather not touch, such as for example a drawing pin, at the location at which the display is imminently to be touched, in order to stop the user from continuing the movement towards the display. The closer the distance, the larger the “menacing object” could be illustrated. Many other intuitive methods of modifying the displayed image are also presented here as embodiments of the present invention.

Another option is to change the size of the displayed features. The features could for example shrink to simulate the notion that the physical display is moving away from the user. Alternatively, the features could be enlarged, in particular abruptly, to give the user the impression that the displayed image is about to “jump out of the screen”, for example causing the user to quickly retract an extended finger. Shrinking or enlarging the displayed image or the user interface can be combined with a three-dimensional monitor in order to give a more complete impression that the image or the user interface is physically moving backwards away from the user or forwards towards the user. Other options include displaying sparks which appear to jump from the display towards the object as it approaches the screen or reducing the colour and/or brightness of the interface in order to reduce the visual quality of the display as a “warning”. Moreover, “old-fashioned” artefacts from the time of LCD displays could be displayed. Each of these effects could be amplified as the distance from the display decreases.

A specific view of the threshold area or the combined threshold-display area, such as for example a side view, could be used to illustrate the extent of approach towards the threshold or display in a highly intuitive way.

Another possible response triggered by the computer is to activate a display moving unit which moves the display away from a gesture or gesturing object which has reached or crossed the threshold or breached a predetermined or selected distance from the display.

The method in accordance with the invention is preferably at least partly executed by a computer, i.e. all the steps or merely some of the steps (i.e. less than the total number of steps) of the method in accordance with the invention can be executed by a computer.

The invention also relates to a program which, when running on a computer or when loaded onto a computer, causes the computer to perform one or more or all of the method steps described herein and/or to a program storage medium on which the program is stored (in particular in a non-transitory form) and/or to a computer on which the program is running or into the memory of which the program is loaded and/or to a signal wave, in particular a digital signal wave, carrying information which represents the program, in particular the aforementioned program, which in particular comprises code means which are adapted to perform any or all of the method steps described herein.

Terms used in the present application are defined in the following.

Within the framework of the invention, computer program elements running on the computer mentioned herein can be embodied by hardware and/or software (this includes firmware, resident software, micro-code, etc.). Within the framework of the invention, computer program elements can take the form of a computer program product which can be embodied by a computer-usable, in particular computer-readable data storage medium comprising computer-usable, in particular computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, in particular a data processing device comprising a digital processor (central processing unit or CPU) which executes the computer program elements, and optionally a volatile memory (in particular a random access memory or RAM) for storing data used for and/or produced by executing the computer program elements. Within the framework of the present invention, a computer-usable, in particular computer-readable data storage medium can be any data storage medium which can include, store, communicate, propagate or transport the program for use on or in connection with the instruction-executing system, apparatus or device. The computer-usable, in particular computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can in particular include a guidance information device which includes means for outputting guidance information. The guidance information can be outputted, for example to a user, visually by a visual indicating means (for example, a monitor and/or a lamp) and/or acoustically by an acoustic indicating means (for example, a loud speaker and/or a digital speech output device) and/or tactilely by a tactile indicating means (for example, a vibrating element or a vibration element incorporated into an instrument).

The method in accordance with the invention is in particular a data processing method using inter alia a computer as a technical means. The data processing method is in particular executed by or on the computer. The computer in particular comprises a processor and a memory in order to process the data, in particular electronically and/or optically. Any calculating steps described are in particular performed by the computer. Determining steps or calculating steps are in particular steps of determining data within the framework of the technical data processing method, in particular within the framework of a program. A computer is in particular any kind of data processing device, in particular electronic data processing device. A computer can be a device which is generally thought of as such, for example desktop PCs, notebooks, netbooks, etc., but can also be any programmable apparatus, such as for example a mobile phone or an embedded processor. A computer can in particular comprise a system (network) of “sub-computers”, wherein each sub-computer represents a computer in its own right. The term “computer” includes a cloud computer, in particular a cloud server. The term “cloud computer” includes a cloud computer system which in particular comprises a system of at least one cloud computer and in particular a plurality of operatively interconnected cloud computers such as a server farm. Such a cloud computer is preferably connected to a wide area network such as the World Wide Web (WWW) and located in a so-called cloud of computers which are all connected to the World Wide Web. Such an infrastructure is used for “cloud computing”, which describes computation, software, data access and storage services which do not require the end user to know the physical location and/or configuration of the computer delivering a specific service. In particular, the term “cloud” is used in this respect as a metaphor for the Internet (World Wide Web). In particular, the cloud provides computing infrastructure as a service (laaS). The cloud computer can function as a virtual host for an operating system and/or data processing application which is used to execute the method of the invention. The cloud computer is for example an elastic compute cloud (EC2) as provided by Amazon Web Services™. A computer in particular comprises interfaces in order to receive or output data and/or perform an analogue-to-digital conversion. The data are in particular data which represent physical properties and/or are generated from technical signals. The technical signals are in particular generated by means of (technical) detection devices (such as for example devices for detecting marker devices) and/or (technical) analytical devices (such as for example devices for performing imaging methods), wherein the technical signals are in particular electrical or optical signals. The technical signals in particular represent the data received or outputted by the computer.

The expression “acquiring data” in particular encompasses (within the framework of a data processing method) the scenario in which the data are determined by the data processing method or program. Determining data in particular encompasses measuring physical quantities and transforming the measured values into data, in particular digital data, and/or computing the data by means of a computer and in particular within the framework of the method in accordance with the invention. The meaning of “acquiring data” also in particular encompasses the scenario in which the data are received or retrieved by the data processing method or program, for example from another program, a previous method step or a data storage medium, in particular for further processing by the data processing method or program. The expression “acquiring data” can therefore also for example mean waiting to receive data and/or receiving the data. The received data can for example be inputted via an interface. The expression “acquiring data” can also mean that the data processing method or program performs steps in order to (actively) receive or retrieve the data from a data source, for instance a data storage medium (such as for example a ROM, RAM, database, hard drive, etc.), or via the interface (for instance, from another computer or a network). The data can be made “ready for use” by performing an additional step before the acquiring step. In accordance with this additional step, the data are generated in order to be acquired. The data are in particular detected or captured (for example by an analytical device). Alternatively or additionally, the data are inputted in accordance with the additional step, for instance via interfaces. The data generated can in particular be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, in particular determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

Advantages, advantageous features, advantageous embodiments and advantageous aspects of the present invention are disclosed in the present description. Different advantageous features can be combined in accordance with the invention wherever technically expedient and feasible. Specifically, a feature of one embodiment which has the same or a similar function to another feature of another embodiment can be exchanged with said other feature. A feature of one embodiment which adds an additional function to another embodiment can in particular be added to said other embodiment.

Embodiments of the present invention will now be described in more detail by referring to the attached drawings.

FIG. 1 shows a schematic illustration of a medical display system in accordance with the invention;

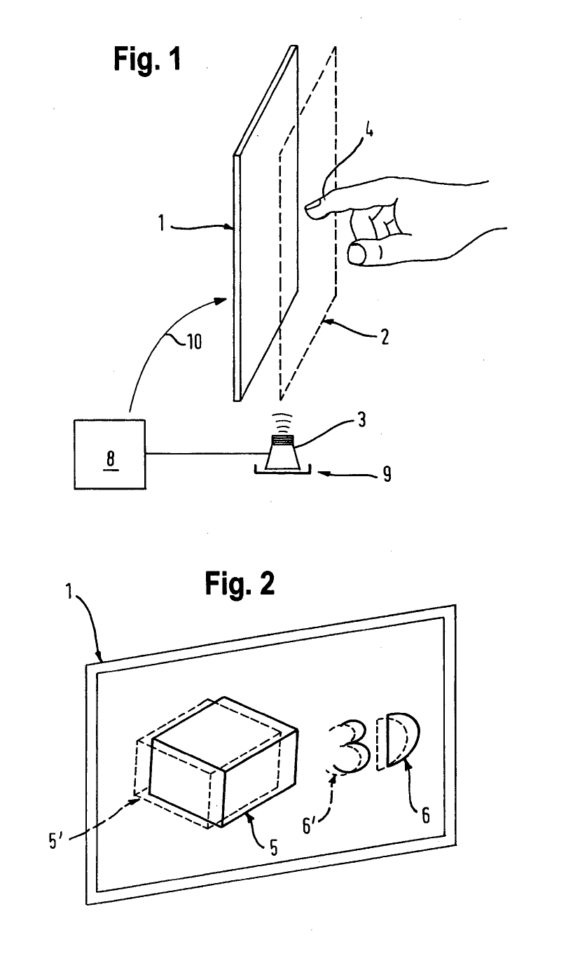

FIG. 2 shows an example of a modification to a displayed image, when a gesture approaches the display too closely;

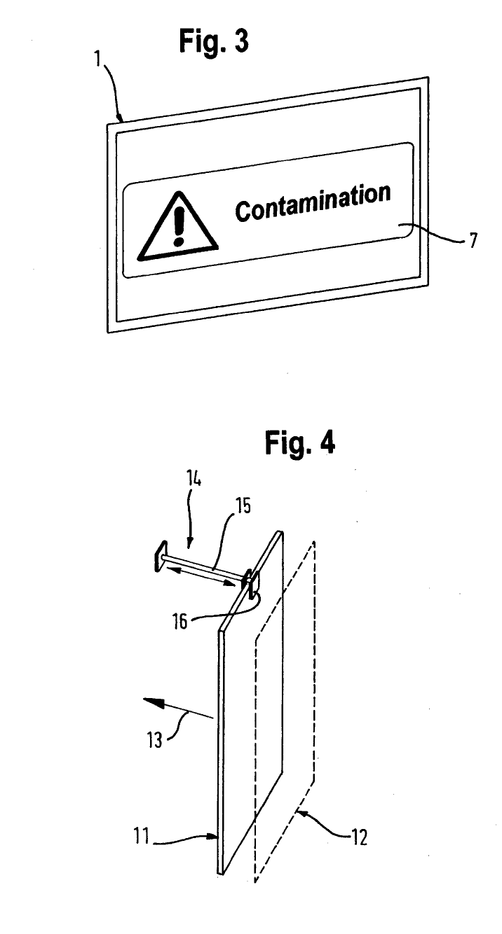

FIG. 3 shows a warning message which can be displayed when the screen (display) is approached too closely or indeed touched; and

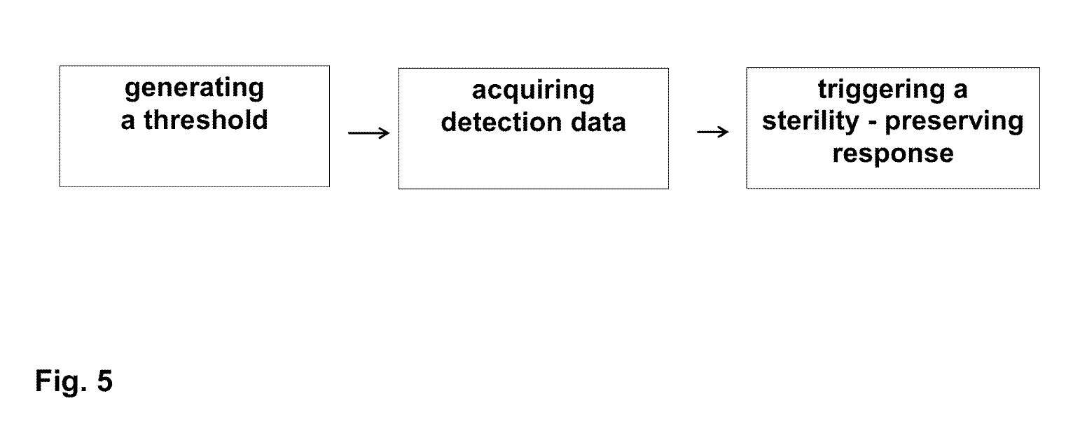

FIG. 4 shows a schematic view of an embodiment of the display system comprising a movable display or screen.

The embodiment of the medical image display system shown in FIG. 1 comprises a display 1 which is also referred to in the following as the screen 1. The screen 1 is an example of a medical display, such as for example the screen of a surgical navigation system for providing image guidance during surgery in an operating theatre. The screen 1 could also be embodied as the screen of a digital light box or any other device which can show medical images and/or user interfaces which are to be operated on or near the screen or any other image display or general medical device which if touched would render the touching person or object non-sterile.

A threshold 2 in front of the screen 1 is shown by a dotted line and is in this example a virtual plane arranged at a certain or predetermined or selected distance from the screen 1. As mentioned above, the threshold 2 can take many forms and can be either actively projected or merely a passively monitored spatial region. It can also have a discernible depth in the direction perpendicular to its main plane of extension and/or the plane of the screen 1 or it can be composed of a number of layered planes.

FIG. 1 schematically shows how the threshold 2 is projected or generated or monitored by a presence detector 3 which operates in one of the ways described above and in particular as described above in the general portion of the present specification. In functional terms, the presence detector 3 monitors whether the threshold 2 is touched or crossed by a gesture or gesturing object, represented in FIG. 1 by an extended finger 4.

A positioning aid is indicated by the reference number 9 and can be a guide rail or receptacle which can be easily positioned at a predetermined distance from the screen 1 by means of simple rules or short bars having a predetermined length. It would of course also be possible to use more sophisticated means of determining the distance between the threshold 2 and the screen 1, such as for example distance-measuring laser devices associated with the presence detector 3. In other embodiments, the presence detector 3 can be fastened to the screen 1 by means of an adaptor or the like and thus positioned in a predetermined manner, such that there is a predetermined distance between the screen 1 and the threshold 2.

The presence detector 3 sends an output signal to a computer 8 if a gesture or gesturing object has reached or crossed the threshold 2, and the computer 8 can then trigger a response. This response can initiate an action or a form of guidance on the screen 1 which is indicated in general terms by the arrow 10 in FIG. 1.

Some of the possible forms of guidance provided to the user of the medical image display system of the present invention are shown in FIGS. 2 to 4. They are triggered by the response of the computer 8 to the threshold being touched or crossed. In other words, the various embodiments of the invention provide feedback to the user of a non-contact gesture control when actual physical contact with a non-sterile or otherwise contaminated screen is imminent. The invention uses gesture tracking in front of the screen and determines the distance between the screen and any physical object performing the gesture. When the physical object (reference number 4 in FIG. 1) approaches the display 1, the image or user interface shown on the display can be changed in such a way as to provide the user with feedback which informs the user that the physical object is getting too close to the screen 1.

In the example shown in FIG. 2, said feedback is provided by simulating an abrupt movement of the displayed image 5, 6 into new positions 5′, 6′ on the screen 1, i.e. the objects on the screen 1 appear to “jump” away from the user if a gesture or gesturing object 4 has reached or crossed the threshold 2, in order to indicate that the gesture or gesturing object 4 has come too close to the screen 1.

Such modifications to the displayed image or user interface can of course take many different forms. The objects 5, 6 could for example be moved so as to appear to “jump” towards the user. Another option is to display a camera view of the screen 1 and the physical object 4 from the side, such that the user can easily see the remaining distance. This is similar to displaying an image as for example in FIG. 1. Another embodiment involves displaying objects which are dangerous to touch, such as for example drawing pins, in order to induce the user to discontinue the movement towards the display 1. The drawing pin(s) could then be shown larger, the closer the gesture or gesturing object comes to the screen 1. The user interface could also be shrunk or enlarged so as to simulate the physical screen moving away from or towards the user. This effect, as also those of any of the embodiments of the invention, can be amplified as the distance to the screen 1 decreases. The virtual shrinking, enlarging or moving (FIG. 2) of the displayed image, as with many of the other possible image modifications, can be enhanced by using a 3D monitor. Sparks could also be displayed on the screen such that they appear to be jumping from the monitor to the physical object 4 approaching the screen 1, and the number and/or intensity of the sparks could increase as the distance decreases.

It is also possible to reduce the colour and/or brightness of the displayed image or the user interface in order to make the screen appear darker, or to display the familiar artefacts which can appear when LED displays are touched. The principle would then apply that the closer the physical object to the screen, the darker the screen or the larger the artefacts.

One important aspect of the invention is that actual contact with the screen 1 is tracked. This can be achieved by for example adding a traditional touch screen interface to the monitor. Alternatively, gesture tracking can be used to determine when actual contact has occurred. In the event of excessive proximity to the screen 1 in general, but in the event of determined actual physical contact in particular (such as might not be sensed or realised by the user), the screen 1 can display a contamination warning 7 as shown in FIG. 3.

The embodiment shown in FIG. 4 comprises a screen 11, a threshold 12 and a display moving unit 14 which in the simple embodiment shown consists of a guide rail 15 with a screen clamp 16 which can be driven by a motor (not shown) to move on the guide rail 15. Alternatively, the screen 11 can be positioned on a movable frame and physically moved backwards, in the direction of the arrow 13, if it is detected that the threshold 12 has been approached or crossed (in the same way as described with respect to FIG. 1).

The physical movement of the monitor should guarantee its sterility in most cases, since it would be necessary to deliberately follow the screen 11 around the room in order to touch it, which the user is hardly likely to do. The physical movement of the screen 1 can also be combined with a visual feedback on the display or user interface, as described above, in order to provide the greatest possible likelihood of preserving the physical object involved in the contact in a sterile state.

1. A feedback system for control or input gestures in a medical environment, comprising:
a presence detector which generates a threshold and acquires detection data which describe whether a medical input gesture crosses the threshold and the extent to which the gesture has passed the threshold; and
a computer which is connected to the presence detector and triggers a sterility-preserving response to a positive detection by the presence detector including information related to a danger level.

2. The feedback system according to claim 1, wherein the presence detector comprises a sensor unit which monitors the presence of an object in a predetermined portion of space which includes the threshold.

3. The feedback system according to claim 1, wherein the presence detector monitors a substantially planar sensing area which includes a threshold plane.

4. The feedback system according to claim 1, wherein the computer is connected to a response indicator which issues a sterility-preserving signal.

5. The feedback system according to claim 1, further comprising a medical display in front of which the threshold is generated.

6. The feedback system of claim 5, wherein the threshold is located at a predetermined or selectable distance from the display which represents a minimum distance to be observed when a gesture or gesturing object approaches the display.

7. The feedback system of claim 5, wherein a positioning aid assists in establishing and/or maintaining a predetermined or selectable distance between the medical display and the threshold and/or between the medical display and the presence detector.

8. The feedback system of claim 5, further comprising a display moving unit for moving the display away from a gesture or gesturing object which has reached or crossed the threshold or breached a predetermined or selected distance from the display.

9. A method of providing sterility-preserving feedback with respect to control or input gestures in a medical system, the method comprising the steps of:
generating a threshold by using a presence detector and acquiring detection data which describe whether a medical input gesture crosses the threshold and the extent to which the gesture has passed the threshold; and
triggering a sterility-preserving response to a positive detection including information related to how dangerous the situation is becoming with regard to non-sterility, by the presence detector by using a computer which is connected to the presence detector.

10. (canceled)

11. The method according to claim 9, wherein the presence detector acquires data which describe the extent to which the gesture or gesturing object has entered or passed the threshold, using one of a continuous, a non-continuous, or in an incremental, distance measurement.

12. The method according to claim 9, wherein the computer's response involves providing user guidance, which comprises one or more of the following:
a warning message, in particular if actual contact with a display has been detected by the presence detector or a touch screen display or a touch detector on the display;
a modification to the displayed image;
a view of the threshold area or a combined view of the threshold and display area which illustrates the extent to which the threshold or display has been approached.

13. The method according to claim 9, wherein the computer's response activates a display moving unit which moves the display away from a gesture or gesturing object which has crossed the threshold or breached a predetermined distance from the display.

14.-15. (canceled)

16. A system, comprising:
a data processing apparatus;
a computer readable storage medium coupled to the data processing apparatus, the computer readable storage medium storing instructions that, when executed by the data processing apparatus, cause the data processing apparatus to perform operations, comprising:
generating a threshold by using a presence detector and acquiring detection data which describe whether a medical input gesture crosses the threshold and further generates an extent to which the gesture has passed the threshold;
triggering a sterility-preserving response to a positive detection including information related to a danger level representative of a level of non-sterility, by using the data processing apparatus being operably connected to the presence detector;
a medical display in front of which the threshold is generated.

17. The system of claim 16 further including instructions to cause the data processing apparatus to provide user guidance including at least one of:
a warning message when actual contact with the display has been detected by the presence detector on the display;
a modification to the displayed image.
